(19) United States
(12) Patent Application Publication
(10) Pub. No.: US 2025/0352193 A1
(43) Pub. Date: Nov. 20, 2025

(54) MEDICAL IMPLANT AND DELIVERY DEVICE FOR A MEDICAL IMPLANT

(71) Applicant: HOLISTICK MEDICAL, Paris (FR)

(72) Inventors: Ellen ROCHE, Galway (IE); Matthew KEILLOR, Paris (FR); Philippe POULETTY, Paris (FR); Antoine PAU, Paris (FR); Marco GARD, Borgomasino (IT); Boris WARNACK, Oberwil (CH); Maëlle BRUNEAU, Paris (FR)

(73) Assignee: HOLISTICK MEDICAL, Paris (FR)

(21) Appl. No.: 19/282,440

(22) Filed: Jul. 28, 2025

Related U.S. Application Data
(63) Continuation of application No. 17/636,595, filed on Feb. 18, 2022.

(30) Foreign Application Priority Data
Aug. 20, 2019 (WO) PCT/IB2019/000967

(51) Int. Cl. A61B 17/00

(52) U.S. Cl. CPC A61B 17/0057; A61B 2017/00004; A61B 2017/00243; A61B 2017/00592; A61B 2017/00606; A61B 2017/00619; A61B 2017/00623; A61B 2017/00628; A61B 2017/00632; A61B 2017/0065; A61B 2017/00907; A61B 2017/00951

(57) ABSTRACT

The invention relates to medical implant (1) that is adapted to close a defect (D) or a cavity, preferably a defect in an atrial or septal wall (W) or a left atrial appendage. The implant (1) comprises an occlusion device (6) and has two states. It is adapted to, in a first state, be deployed to a defect site (D), where it can be brought into a second state by an activation mechanism. It is adapted to close said defect (D) in said second state.

### CROSS-REFERENCE TO RELATED APPLICATIONS

This application is a continuation of U.S. patent application Ser. No. 17/636,595 (filed 18 Feb. 2022), which is a National Stage completion of International application PCT/EP2020/073175 (filed August 2020), which claims priority to International application PCT/IB2019/000967(filed 20 Aug. 2019). All subject matter set forth in the above referenced applications is hereby incorporated by reference in its entirety into the present application as if fully set forth herein.

### FIELD OF THE INVENTION

The present invention relates to a medical implant and method for implanting a medical implant according to the preamble of the independent claims.

### BACKGROUND OF THE INVENTION

Defects in tissue, for example atrial septal defects (ASD) or ventricular septal defects (VSD), are a fairly common condition in humans that is typically treated minimally invasively. Such defects can cause a variety of symptoms such as shortness of breath and a higher burden on the heart and lungs.

Similar conditions may arise from defects caused by imperfect fitting of implants, for example paravalvular leaks in artificial heart valves.

Cavities in a human body, even when physiologically normal, can cause medical conditions. For example, blood clots can form in the left atrial appendage (LAA) and can cause conditions such as strokes, in particular in patients with atrial fibrillation.

As a consequence, a myriad of implantable devices has been proposed in the prior art, many of which can be deployed in a minimally invasive way.

For example, closure of an atrial septal defect (ASD) by deploying umbrella-like implants through a catheter has been disclosed by Lock et al. (DOI: 10.1161/01.CIR.79.5.1091).

Similarly, EP 2 688 486 discloses an expandable device for closing a septal defect.

However, the known devices have several disadvantages. They usually have a pre-determined size and have little flexibility to be adapted to the individual needs of a specific patient. If an implant fits poorly at the implant site, however, an increased risk for complications can arise for the patient.

### SUMMARY OF THE INVENTION

Thus, the object of the present invention is to overcome the drawbacks of the prior art, in particular to provide a medical implant that can be better adapted to a specific patient's need and that lowers the risk of complications due to poor fit.

This and other objects are achieved by the medical implant and the methods according to the characterizing portion of the independent claims of the invention.

A medical implant according to the invention is adapted to close a defect or a cavity, in particular a defect in an atrial or septal wall, or a left atrial appendage. It comprises an occlusion device. Furthermore, it comprises a first state and a second state. In the first state, it is adapted to be deployed to a defect site, in particular a defect site at an atrial or septal wall or a left atrial appendage. It can be brought into second state by an activation mechanism and is adapted to close such a defect or cavity in the second state. The difference between the first and the second state may be a difference in shape, and/or it may comprise the release of an adhesive, or of an active substance. Additionally or alternatively, it may also comprise an expansion of the implant material, in particular a self-expansion due to exposure to an increased temperature and/or water and/or release of a physical constraint.

In some embodiments, the implant may comprise a helical anchor connected to a self-expanding foam plug or tampon. The helical anchor may, in particular, provide a constant attachment force. Such a helical anchor is particularly advantageous because it reduces the risk of dislodgment, may provide attachment to different tissues, and does not require activation. Such an anchor may also be used as a temporary attachment means.

Additionally or alternatively, the implant may comprise a porous mesh or a porous capsule, in particular a capsule or a mesh comprising or consisting of polyurethane. The implant may be filled by transcatheter injection with a liquid, preferably a liquid polymer and/or adhesive, preferably a liquid polymer and/or adhesive that can later be cured by a mechanism such as photoactivation or other chemistry. The implant may be adapted to release the liquid if the pressure in the adhesive reaches a certain threshold.

The implant may further comprise a self-sealing plug, in particular a silicone plug. Such a plug may assist withdrawal of delivery and/or treatment device, for example before an adhesive is entirely cured. The plug may further contain the liquid in the capsule.

The implant may comprise an electrospun expandable structure, in particular expandable by a compliant balloon. The expandable structure may be coated and/or impregnated with methacrylated gelatin (GelMA) and/or a tissue adhesive. Additionally or alternatively, a bioadhesive may be used. The GelMA and/or the adhesive may be released if the structure is stretched. Electrospun structures are advantageous as they are easy to manufacture, conformable, and provide suitable tissue ingrowth potential.

The occlusion device is, in particular, adapted to be brought into the defect/cavity itself and plug the defect/cavity. It may be adapted to the particular size and shape of the defect/cavity to be occluded, in particular to the individual size and shape of a defect in an individual patient. In particular, it may be adapted to apply a radial outward force on the defect wall.

A first and a second state in particular enables an easier deployment to an implant site. For example, the implant may have smaller size, a more suitable shape, or an inactivated state of an adhesive comprised in it that allows for an easier deployment in a minimally invasive way.

Preferably, the medical implant comprises at least one peripheral disk. Even more preferably, it comprises at least two peripheral disks. Such disks can be adapted to on either side of the defect. The peripheral disks can increase the sealing effect of the occlusion device. Additionally or alternatively, the peripheral disks can also provide fixation to the implant site through any fixation means known in the art. For example, the occlusion device may be attached to a peripheral disk mechanically through a wire, a suture, or a strut, or it may be attached by means of an adhesive, or it may be formed integrally with the peripheral disk.

Preferably, at least one peripheral disk is adapted to mechanically anchor the occlusion device. Even more preferably, at least two peripheral disks are adapted to mechanically anchor the occlusion device. This provides increased safety upon implantation, because the peripheral disk cannot easily be transferred through the defect. Thus, the occlusion device is secured in the defect. In particular, if one disk on each side of the defect is used, the occlusion device is prevented from accidentally, for example due to poor fitting, leaving the defect.

Preferably, the occlusion device is connected or connectable to at least one peripheral disk by an interconnector. In particular, the interconnector may be at least one of a group of a suture, a wire, and a strut. Interconnectors provide increased mechanical strength such that the occlusion device is prevented from accidentally leaving the defect. It is particularly advantageous if the implant comprises two peripheral disks and the occlusion device is mechanically connected to both peripheral disks. In this case, occlusion device is securely fastened in the defect.

Preferably, the medical implant comprises a braided structure. In particular the braided structure may be made of Poly-L-Lactic acid and/or Poly-Lactic-co-Glycolic Acid. Implants made of these materials can be biodegradable and disintegrate in the human body.

The implant may be further adapted to have a substantially elongated shape in its first state, and comprise two disks that extend orthogonally from its longitudinal axis in its second state. This allows for an easy deployment in a minimally invasive way, for example by means of a catheter, because the shape of the implant in the first state may be substantially linear. In the second state, the two disk-like arrangements provide substantially the same type of mechanical anchoring as the peripheral disks also described herein. The braided structure is particularly advantageous in that it provides a structure that can easily be brought into either shape. Additionally, a wide variety of shapes, for example larger or smaller disks, or different numbers of disks, can be achieved easily. In this context, a disk shall be understood as any substantially flat shape that is oriented orthogonally from to the longitudinal axis. It shall not, however, be understood as limiting in any way in terms of the two-dimensional shape, for example to a round shape, in the plane orthogonal to the longitudinal axis. For example, a disk may have a star shape, or a triangle shape, or a square shape. In addition, the braided structure may also support a foam with an adhesive composition.

Poly-L-Lactic acid and/or Poly-Lactic-co-Glycolic Acid are particularly advantageous materials because they are biocompatible and thus unlikely to trigger negative reactions by the body, for example inflammations or allergies. In addition, they can be adapted to be biodegradable. Thus, the implant can also be adapted such that a part of the implant or the entire implant degrades in the human body over a certain time frame. This is particularly advantageous if a part of the implant is only needed during deployment, for example to provide anchoring, but cannot easily be removed from the body. However, the implant may of course consist of other materials, in particular other biocompatible polymers.

Preferably, the medical implant comprises at least one radio-opaque or echogenic marking, preferably a cuff. In particular, it may be located along a longitudinal axis of the medical implant. This allows an operator to easily detect different positions of the implant. For example, if a radio-opaque marker is arranged along the longitudinal axis of the implant on both ends of the occlusion device, the operator can easily determine if and when the occlusion device is properly arranged within the defect. Similarly, they could be arranged at the ends of two peripheral disks or other disk-like structure. Of course, it would also be conceivable to place additional radio-opaque markers that allow for determination of the deployment state. For example, they could be placed on expandable parts of a braided structure such that the operator can track how far the disk-like structure extend from the longitudinal axis.

Preferably, the braided structure comprises at least one inner cavity that is at least partially filled with an adhesive. The inner cavity may also be completely filled with the adhesive. Preferably, the adhesive is a dehydrated adhesive. The cavity may be located on the inside of the braided structure. This allows for the deployment of an adhesive composition at the implant site, leading to enhanced fixation of the medical implant, in particular of the occlusion device, at and/or in the defect. The adhesive may be a light-curable adhesive, and/or a dried adhesive, and/or a dehydrated adhesive. It may be particularly adapted to be activatable by exposure to water, humidity, or preferably blood. Of course, it would also be possible to adapt the adhesive to be activatable by exposure to other substance, preferably other substances that are present in the human body. The cavity may be a pocket on the inside surface of the braided structure, or it may fill the entire inner volume of the braided structure. Additionally or alternatively, the cavity may also be arranged in the fibers of the braided structure.

Preferably, the medical implant comprises an adhesive composition, wherein in the first s state, the adhesive composition is contained in the medical implant. The medical implant may be adapted to release the adhesive composition in its second state. This provides a particularly safe way to transfer the adhesive to the implant site because it is not exposed to the body or bodily fluids until it reaches the implant site. In particular, the medical implant may be adapted such that the adhesive composition can be selectively released, meaning that the operator can release the adhesive composition at his discretion, by actuation of a trigger. The trigger may, in particular, be the inflation of a balloon. Of course, other mechanisms to selectively release the adhesive are possible, in particular mechanical, thermal and/or chemical mechanisms.

Preferably, the medical implant comprises at least one cavity. The adhesive composition is disposed in the at least one cavity. The cavity is adapted to selectively release the adhesive composition. For example, the cavity may be adapted to burst upon applying of a mechanical deformation or pressure, or to degrade upon exposure to electromagnetic radiation such as UV/visible light.

Preferably, the medical implant is adapted such that the adhesive composition is released upon mechanical deformation, in particular mechanical compression. In the context of minimally invasive implantation of an implant, mechanical deformation is a particularly advantageous way for such a release, because it can easily be applied, for example by a balloon catheter. In particular, the adhesive may be arranged close to the surface of the medical implant, in particular the occlusion device, such that it can be released by inflation of an inner balloon arranged on the inside of the medical implant that squeezes out the adhesive.

Preferably, the at least one peripheral disk, in particular the at least two peripheral disks, are adapted to selectively apply a mechanical deformation, in particular a mechanical pressure, to the occlusion device. In particular, an interconnector may be adapted to pull the peripheral disks such that it applies a pressure and/or deformation to the occlusion device. Preferably, this pressure and/or deformation releases the adhesive.

Preferably, the medical implant comprises at least two cavities. In particular, the at least two cavities may be arranged in proximity to the outer surface. The adhesive composition comprises at least two components that are individually disposed in the at least two cavities. Thus, the two components are separated from each other and not mixed in the medical implant. However, this allows for mixing upon release of the adhesive composition. Proximity shall be understood as a distance that is short enough such that the adhesive can penetrate to the surface of the implant. In particular, it could be understood as less than two, preferably one, cavity diameter away from the surface. Additionally or alternatively, it could also be understood as a distance less than 1 mm, preferably less than 0.5 mm, even more preferably less than 0.1 mm. The cavities can be arranged substantially on the entire surface of the medical implant or the occlusion device, or only on a part of the implant. They may be arranged on a distal or a proximal end of the occlusion device or only one a part of the circumference.

Preferably, the adhesive composition is adapted to be curable by mixing of the at least two components. Alternatively, the adhesive composition may be adapted to spontaneously cure upon mixing of the two components. Such an adhesive composition comprising at least two components increases the safety of the implant, because curing can be better controlled. In particular, accidental curing, for example by exposure to light before implantation, can be avoided.

Preferably, the adhesive composition is adapted to be gradually curable, preferably cross-linkable, in particular by exposure to electromagnetic radiation such as visible light, infrared light, ultraviolet light, or X-rays. Gradual curing shall be understood a continuous progress in the curing, wherein the mechanical strength continually increases with the degree of curing and/or cross-linking. For example, this may be achieved by an increase of the cross-linking in the adhesive composition, in particular by exposure to UV light, visible light, IR light, and/or X-rays. This allows for a tuning of the mechanical properties of the implant. For example, the adhesive composition could be cured to a lesser degree if some elasticity is required at a certain implant site. By contrast, if a harder material is required, the curing could be performed to a higher degree. Additionally or alternatively, the degree of cross-linking can be controlled by modulating the molecular weight of the prepolymer and/or the degree of functionalization with cross-linkable functional groups.

Preferably, the adhesive composition is adapted to be curable by exposure to electromagnetic radiation, in particular UV light, IR light, visible light, and/or X-rays. This is particularly advantageous in combination with a catheter comprising a light guide, for example a catheter as disclosed in WO 15/175662. In particular, the occlusion device may also be adapted such that an optical fiber can be transferred and/or withdrawn through it.

Preferably, the medical implant comprises, even more preferably consists of, an expandable material. In particular, the expandable material may be a shape-memory material, for example, an alginate-based shape memory material. Any part of the medical implant could be made of an expandable material, but it is particularly advantageous to make at least one of the occlusion devices in the defect and a peripheral disk out of and expandable material. This allows for minimizing the cross-section orthogonally to the longitudinal axis of the medical implant for easy implantation with a catheter. Of course, it would also be possible to arrange an expandable material at another location in the medical implant, and/or to cover it with another material such that the expandable material simply provides the volume expansion. Expansion at the implant site then provides the desired shape. Particularly well-suited materials are hydrogels, alginate-based cryogels, collagen-based materials, gelatin-based materials, and other biodegradable materials. In particular, the medical implant may also comprise or consist of a 3D-printed or cast material.

Preferably, the shape memory material is adapted to expand upon deployment and occlude said defect upon expansion and occlude the defect upon expansion. The diameter in a direction orthogonal to the longitudinal axis of the implant may be less than 3 mm, preferably less than 2 mm, even more preferably less than 1 mm before expansion, and comprised in a range of 3 to 8 mm, preferably 4 to 7 mm, even more preferably 5 to 6 mm, after expansion.

Preferably, the medical implant comprises a central diameter, and two peripheral diameters. Preferably, the central diameter is smaller than the peripheral diameters, such that the medical implant comprises a dumbbell shape. Alternatively, the medical implant could also only have one peripheral diameter that is larger than the central diameter and as such comprise a mushroom shape. This enables self-centering of the medical implant, wherein the implant will automatically be pushed in the center of the defect due to its shape.

Preferably, the occlusion device comprises pericardial tissue, in particular arranged on the outer surface of the implant. Pericardial tissue provides superior biocompatibility. Thus, it is preferably arranged such that is in contact with the tissue surrounding the medical implant. In particular, it may be bonded into the defect by an adhesive, in particular a glutaraldehyde-based bioadhesive. It may also be cross-linked with native tissue.

Preferably, the occlusion device comprises a material that is flexible in its first state and becomes stiff in the second state, in particular upon exposure to electromagnetic radiation, in particular UV light, IR light, visible light, and/or X-rays.

Preferably, the medical implant comprises a distal end that is stiffer than a proximal end. The distal end may be adapted to serve as a cap towards the left atrium. Alternatively, a proximal end may serve as a cap towards the right atrium.

Preferably, the medical implant comprises a dead-end cavity, or a blind hole, in particular along its longitudinal axis, that is adapted to receive a balloon catheter. This allows for a balloon catheter to assist an expansion of the medical implant, and/or to release an adhesive. It may also assist the curing of a photocurable adhesive composition by providing a possibility to distribute light evenly along the longitudinal axis of the medical implant.

Preferably, the medical implant is self-expanding. It may or may not be comprise a self-expanding material, and may also be self-expanding by expansion of an expandable structure.

Preferably, the medical implant is at least partially transparent to light, in particular to visible light, ultraviolet light, and/or infrared light, in particular along the longitudinal axis of the implant. This allows for a transfer of light through the implant, for example to provide curing of an adhesive composition, or for signal transmission, or for heating. The transparency may be provided by a transparent material, by a partially hollow shape, or a combination of the two. In particular, the medical implant may be a braided structure with a transparent material arranged on the inside. Additionally or alternatively, a photocurable adhesive on a surface of the implant may activated via light transmission by the transparent material for use as a filler.

Preferably, the medical implant has one of a size and a shape that at least partially substantially corresponds to the size of a human left atrial appendage. The size of the implant may also entirely correspond to the size of a human left atrial appendage.

Preferably, the implant has a first and a second section along a longitudinal axis, wherein the first section has a larger cross-section in plane perpendicular to the longitudinal axis than the second section. In particular, the implant may have a mushroom of half-dumbbell shape.

Preferably, the medical implant has a flat or planar shape. Flat may in particular be understood as being substantially larger in two dimensions compared to a third dimension. The shape may be curved and/or inclined, in particular with respect to a plane perpendicular to the short dimension. The shape of the implant is this plane may in particular be of substantially a half-moon shape, for example to close a paravalvular leak.

Preferably, the implant has at least one section with a diameter in the range of 10-25 mm, particularly preferably 15-20 mm.

The invention is further directed to a method of closing a cavity in a patient, in particular a left atrial appendage. The method comprises the step of closing the cavity with an implant as described herein.

The invention is further directed to a method of closing a defect in a patient, in particular a defect in an atrial or septal wall. The method comprises the step of closing the cavity with an implant as described herein.

The invention is further directed to a method of treating a paravalvular leak in a patient. The leak is formed by an opening between a valvular implant and the patient's tissue. The method comprises the step of at least partially closing the opening with a medical implant as described herein. Preferably, the opening is completely closed.

The invention is further directed to a method of producing a medical implant. In particular, the implant may be an implant as described herein. The method comprises the step of imaging an area to be treated. Preferably, the area to be treated comprises or consists of an opening, a defect, or a cavity, in particular a left atrial appendage or patent foramen ovale. A further step comprises determining at least one of a size and a shape of the area to be treated. The method further comprises the step of designing the implant such as to have the same size and/or shape as the area to be treated. Alternatively, the size and shape may be the size and shape multiplied by a factor, respectively.

The invention is further directed to a method of closing a cavity in a patient, in particular a left atrial appendage.

The method comprises the steps of:

Sealing an area outside of the cavity, in particular an entrance of the cavity, in particular an ostium,

Applying an negative pressure to the cavity such as to collapse the cavity,

At least one of the following steps:
a. Filling the collapsed cavity with an adhesive, in particular such as to re-expand the cavity to its original volume;
b. Permanently sealing the collapsed cavity.

The invention is further directed to a treatment device for treating, in particular closing, a cavity in a patient. The cavity may in particular be a left atrial appendage. The device comprises a sealing member adapted to at least temporarily seal the cavity. The sealing member may in particular comprise or consist of a balloon and/or an expandable disk. The device further comprises a fluid transmission line adapted to at least apply an negative pressure and/or transport a fluid to an area distal of the sealing member such as to collapse a cavity. In particular, the fluid transmission line may be connected to a vacuum line and/or a fluid reservoir, for example a reservoir of a saline buffer solution (such physiological buffer saline).

The device may, preferably, comprise several separate fluid transmission lines for simultaneous sucking and transport of another fluid.

The invention is further directed at a delivery device, in particular a catheter device, that carries a medical implant as described herein. In particular the catheter device may be particularly adapted for the medical implant by providing an expansion mechanism and/or a curing mechanism. For example, the catheter device may be adapted to transmit light and/or heat.

Additionally or alternatively, the delivery device may be adapted to deliver an adhesive to an opening, a defect, or a cavity, for in-vivo formation of an implant. The delivery device may comprise a mixing chamber, in particular for mixing of a two-component adhesive.

Any delivery device disclosed herein may additionally comprise at least one balloon, preferably two balloons, to create a casting mold for an adhesive. Particular preferably, the delivery device comprises two balloons and a transmission line that opens in an area in between the two balloons such as to transport an adhesive composition in said area. The delivery device may also comprise a light transmission guide for example an optical fiber, for delivering electromagnetic radiation to a treatment site. Electromagnetic radiation may in particular include UV light, visible light, IR light, and/or X-rays.

To that end, the invention is also directed to a method comprising the step of turning a patient such that the opening, which preferably is formed as a blind hole, opens in a direction opposite a direction of a gravitational force, and a second step of filling the opening with an adhesive using a delivery device as described herein. The adhesive may have a density that is higher than the density of blood. Alternatively, the patient may be turned such that the opening opens in a direction of a gravitational force, in particular if the adhesive has a density lower than the density of blood.

The implant according to the invention may additionally or alternatively comprise or consist of a mesh or a braid coated with a hydrogel.

### BRIEF DESCRIPTION OF THE DRAWINGS

In the following, the invention is described in detail with reference to the following figures, showing:

FIG. 1: a schematic embodiment of a medical implant in a side view.

FIG. 2: an embodiment of a medical implant with a catheter device.

FIG. 3: an expandable element for a medical implant.

FIG. 4a-4b: an embodiment of a medical implant from two different perspectives.

FIG. 5a-5b: another embodiment of a medical implant in two different perspectives.

FIG. 6: another embodiment of a medical implant in a first, collapsed state.

FIG. 7: the embodiment of a medical implant of FIG. 6 in a second, expanded state.

FIG. 8a-8b: another embodiment of a medical implant in a first and a second state.

FIG. 9: another embodiment of a medical implant.

FIG. 10: an occlusion device for a medical implant.

FIGS. 11a-11b11d: schematically an occlusion of a cavity using a device.

FIGS. 12a-12c: schematically an occlusion of a cavity using an implant.

FIGS. 13a-13d: schematically an occlusion of a cavity using an alternative implant.

FIG. 14: schematically an alternative implant being used to occlude a cavity.

FIG. 15: schematically an implant being used to occlude a cavity.

FIG. 16: schematically an implant being implanted into a cavity.

FIG. 17: schematically an implant in a cavity.

FIG. 18: schematically an alternative implant in a cavity.

FIG. 19a-19b: schematically alternative embodiments of implants adapted to occlude a cavity.

FIG. 20: an alternative implant for occlusion of a cavity.

FIG. 21: schematically a treatment device.

FIGS. 22a-22g: schematically a method of occluding a cavity.

FIGS. 23a-23g: schematically an alternative method of occluding a cavity.

FIGS. 24a-24b: schematically the implant of FIGS. 23a-23g.

### DETAILED DESCRIPTION OF THE PREFERRED EMBODIMENTS

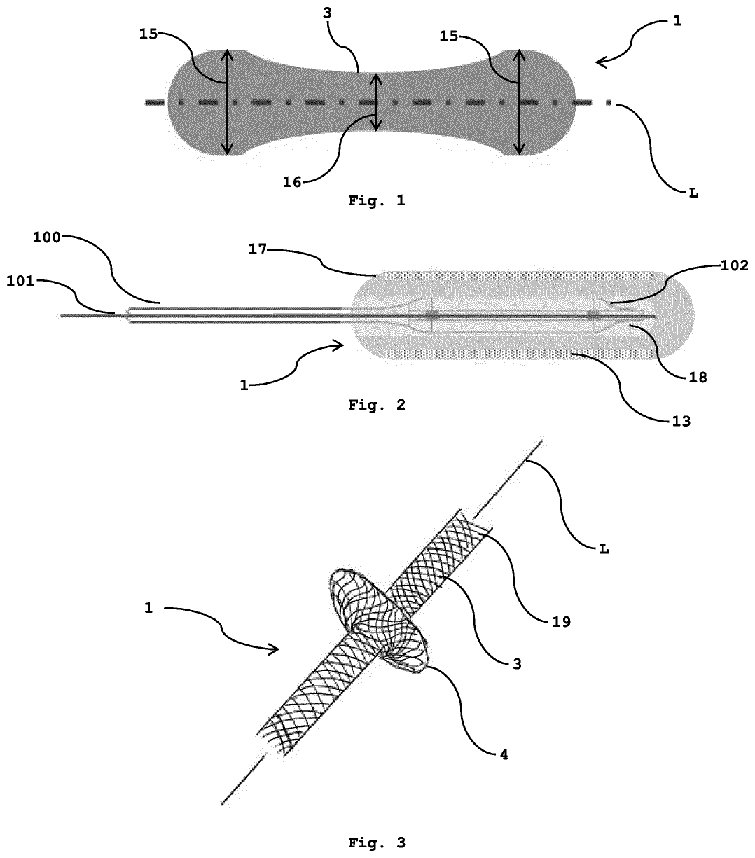

FIG. 1 shows schematically a medical implant 1 according to the invention. It is made of a self-expandable material, in this case an alginate-based shape memory material. In this simple embodiment, the occlusion device 3 is substantially the entire implant. It comprises, however, a central diameter 16 along the longitudinal axis L and two peripheral diameters 15. The central diameter 16 is smaller than the peripheral diameter 15. Thus, the medical implant 1 has an hour glass, or dumbbell-like, shape. This provides for self-centering in a septal or atrial defect (not shown) at the implant site such that the defect is closed. Here, the implant is only shown in its second state that it would be in upon implantation.

FIG. 2 shows a medical implant 1 in combination with a catheter device 100. The catheter device 100 comprises a balloon 102 and an optical fiber 101. The medical implant has an elongated dead-end cavity 18 inside of the implant that is adapted for the insertion of the balloon 102 of the catheter device 100. The medical implant further comprises an area close to its surface 17 that has a plurality of cavities 13. These are filled with an adhesive composition. Here, the adhesive composition only comprises one component and is curable by exposure to visible light. The medical implant 1 can be brought to an implant site by means of the catheter device. At the implant site, the balloon is inflated such that the medical device 1 inflates. The mechanical deformation inflicted by the inflated balloon squeezed out the adhesive composition from the cavities 13. The adhesive composition is then cured by means of exposure to visible light that is transmitted by the optical fiber 101. This permanently fixes the medical implant 1 in the defect (not shown) by the adhesion of the adhesive composition. Thus, the catheter device 100 can be retracted and the septal defect that is treated here is closed. Because the adhesive composition is sufficiently strong to retain the medical implant 1 in the defect, a change in the shape is not necessary to achieve the desired effect. However, it would of course be possible to adapt the medical implant to also change its shape, for example to a shape as shown in FIG. 1. Here, the medical implant is further adapted such that occlusion device 6 itself is also curable by exposure to visible light. Thus, during the curing of the adhesive composition, the occlusion device 6 becomes mechanically stiffer. Thus, it is elastic before and during deployment. However, upon termination of the treatment, the entire medical implant 1 is stiff.

FIG. 3 shows schematically a medical implant 1 comprising braided structure 3. Here, the medical implant 1 is shown in its second state where it comprises a disk structure 4. The disk structure is not present in the first state and arises from expansion in a direction orthogonal to the longitudinal axis L. Thus, in the first state, the shown embodiment of the medical implant 1 had an elongated shape. The braided structure 3 comprises fibers 18 made of biodegradable poly (lactic-co-glycolic acid) and poly (lactic acid). Thus, the braided structure 3 may act as a scaffold for tissue ingrowth. It would of course be possible to use fibers of only one of the two materials as well. Alternatively, the braided structure 3 may comprise or consist of a material that is not biodegradable. Furthermore, the braided structure 3 has a hollow shape, making it is transparent to visible light along its longitudinal axis L. On the inside of the braided structure, not visible in this illustration, a dehydrated adhesive is arranged on the surface of the braided structure. The adhesive composition is adapted to self-expand upon exposure to humidity inside the human body. Thus, it can fill a substantial part of the inside of the braided structure and is then curable by exposure to visible light. The adhesive composition, upon expansion and curing, closes the defect. The braided structure is then no longer needed and degrades in the human body within a week.

FIGS. 4a and 4b show an embodiment of a medical implant 1 comprising one peripheral disk 5 that is mechanically connected to an occlusion device 6. The mechanical connection provided by a wire 12 that is permanently attached to both the occlusion device 6 and the peripheral disk 5. The wire does not penetrate the peripheral disk 5 and is thus not visible on the side of peripheral disk facing away from the occlusion device 6 as shown in FIG. 4a. Here, the medical implant 1 comprises only one peripheral disk, but it would of course be possible to arrange more than one peripheral disk at the occlusion device 6 in the same way. The occlusion device 6 is made of an expandable material, while the peripheral disk 5 is made of a non-expandable, biocompatible polymeric material. The wire connecting 12 the peripheral disk 5 and the occlusion device is made of Nitinol, but it could of course also be made of another biocompatible metallic material, or even a polymer.

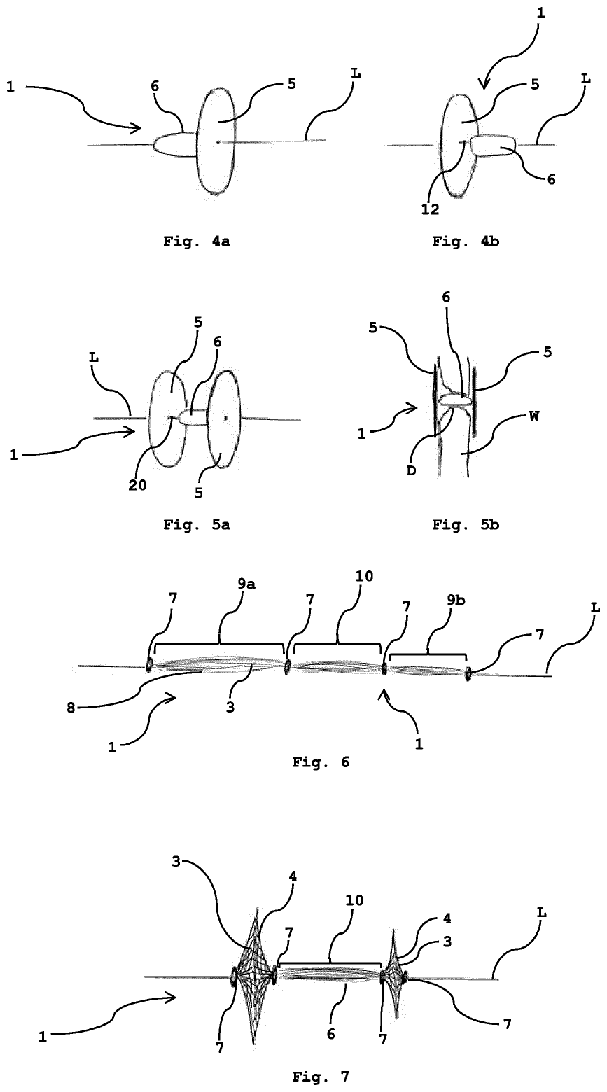

FIG. 5a shows an embodiment of a medical implant similar to the one illustrated in FIGS. 4a and 4b. In this embodiment, the medical implant 1 comprises two peripheral disks 5. They are connected by a mechanical interconnector 20 that also provides attachment to the occlusion device 6 and is oriented along the longitudinal axis L of the medical implant 1. The occlusion device 6 comprises a plurality of cavities (not shown) with an adhesive composition arranged therein. The adhesive composition has two components and is adapted to spontaneously cure upon mixing.

FIG. 5b shows the medical implant 1 as shown in FIG. 5a when implanted to close a defect D in an atrial wall W. The occlusion device is arranged within the defect D. The peripheral disks 5 are arranged on both sides of the wall W and the defect D. The interconnector 20 is adapted to pull together the peripheral disks and is not visible anymore in this illustration. It exerts a force along the longitudinal axis L such that the occlusion 6 device is compressed and anchored in the defect. This releases the adhesive composition from the plurality of cavities (not shown). The two components of the adhesive spontaneously mix and cure, thus completely sealing the defect D.

FIG. 6 shows an embodiment of a medical implant 1 comprising a braided structure 3. The braided structure 3 is disposed as a collapsed structure 8 in this illustration. The medical implant further comprises four radio-opaque cuffs 7 that divide the medical implant into three parts 9a, 9b, 10. The medical implant has, due to the collapsed braided structure 8, a substantially linear shape that is particularly advantageous for minimally invasive implantation.

FIG. 7 shows the same embodiment as shown in FIG. 6 but after expansion of the two peripheral parts 9a, 9b of the braided structure that form disk-like structures 4. The central part 10 of the implant remains collapsed and forms the occlusion device 6 in this embodiment. The occlusion device is mechanically flexible in this example and can comprise a dehydrated adhesive on the inside of the structure (not visible). The dehydrated adhesive is adapted to swell upon exposure to blood and is then curable by exposure to ultraviolet, infrared, and visible light.

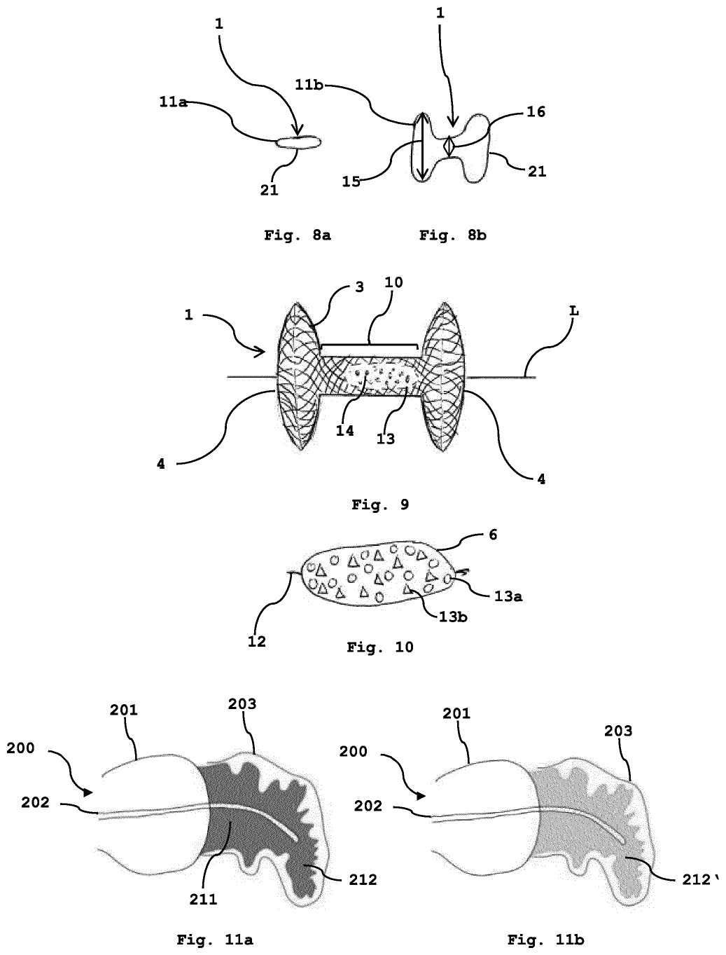

FIGS. 8a and 8b show an embodiment of a medical implant 1 made of an alginate-based shape memory material. In FIG. 8a, the implant is in its first state 11a and has an elongated shape.

As shown in FIG. 8b, upon transport to an implant site, the implant 1 can be brought into its second state 11b, where it self-expands and reaches a dumbbell shape with a central diameter 16 that is smaller than the peripheral diameters 15. Through the expansion, the medical implant 1 can occlude a defect. The medical implant shown in FIGS. 8a and 8b is coated with a layer of pericardial tissue 21 providing superior biocompatibility.

FIG. 9 shows another embodiment of a medical device 1 comprising a braided structure 3. It comprises two peripheral parts 4 that extend away from the longitudinal axis L and a central part 10 that is adapted to be placed in a defect. Here, the central part 10 of braided structure is not collapsed as in other embodiments, but instead exhibits a tubular shape. A part of the braided structure has been made transparent to show a cavity 13 in the inside of the tubular part 10. The cavity comprises an adhesive composition that is gradually curable by exposure to visible light. With increasing degree of curing, its mechanical stiffness increases. Thus, this embodiment is particularly advantageous if, for example, an operator intends to arrange the medical implant 1 such as to have a distal end that is stiffer than a proximal. This can, for example, provide cap towards the left atrium. However, it would of course also be possible to cure any other part of the implant for a longer period of time such that it becomes stiffer. Similarly, it could also be completely cured throughout such that the mechanical properties were homogenous after curing.

FIG. 10 shows in more detail an occlusion device 6 as with two different types of cavities 13a, 13b. Here, the different cavities 13a, 13b are shown with different shapes for clarity, but of course it is not necessary to adapt them in that way. It would also be conceivable to arrange them with identical shape but different sizes, or identical shapes and identical sizes. Two different components of an adhesive composition can be arranged in these cavities. Here, the cavities are adapted to degrade upon exposure to ultraviolet light. Therefore, exposure to ultraviolet light releases the adhesive composition of the occlusion device 6 and its cavities 13a, 13b. Alternatively, any other electromagnetic radiation disclosed herein may be used for degradation. The occlusion device 6 also comprises a wire to be connected to a peripheral disk. The shown embodiment of the occlusion device 6 is similar to the occlusion device shown in FIGS. 5a and 5b with the difference that a differently adapted adhesive composition is comprised in the cavities 13a, 13b.

FIGS. 11a to 11d schematically show a method of occluding a cavity 203, in the present case a left atrial appendage LAA, using a device 200 according to the invention.

FIG. 11a shows a first step of the method. A device 200 comprising a sealing member 201 is formed by a balloon, through which a transmission line 202 extends to a distal area of the device 200. The balloon 201 is placed such as to temporarily close and seal the cavity 203. The transmission line 202 is in fluid communication with the inner volume of the cavity 203. In the step illustrated here, the cavity 203 is filled with blood 212.

FIG. 11b shows a second step of the method, wherein the device 200 is arranged substantially as shown in FIG. 11a. The cavity 203 is filled with a saline buffer solution 212′ that has been flushed into the cavity via the transmission line 202. In the shown embodiment of the device 200, the transmission line 202 serves both to transport liquids to the cavity 203 and to remove liquids therefrom. Alternatively, the device 200 may comprise two or more separate transmission lines for removing blood 212 and/or other liquids, and for transporting fluids. In the present case, a portion of blood 212 was removed through the transmission line 202, and subsequently an equivalent amount of saline buffer solution injected into the cavity 203. This fluid replacement can be repeated until the cavity is entirely filled with saline buffer solution, as shown here. Alternatively, other liquids may be employed as well.

FIG. 11c shows the cavity 203 which is filled with a liquid adhesive 212′. The adhesive 212′ is presently configured as 1-component adhesive that cures and hardens when exposed to body temperature. Alternatively, two-component or multicomponent adhesives are conceivable as well. Additionally or alternatively, the adhesive may be curable by exposure to light, humidity, and/or other curing mechanisms known in the art. The saline buffer solution can be exchanged with adhesive in substantially the same way as described in the context of the exchange of blood 212 with saline buffer 212′ in FIG. 11b above. It is also conceivable to directly exchange blood 212 with adhesive 212″ without intermediate rinsing with saline buffer solution 212′. Presently, the transmission. line 202 may be retracted relative to the balloon 201 while the balloon remains in place such as to seal the cavity 203 while the adhesive 212″ hardens.

FIG. 11d shows the cavity 203 after removal of the device 200. The adhesive 212″ is hardened and is adhesively bound to an inner wall of the cavity 203.

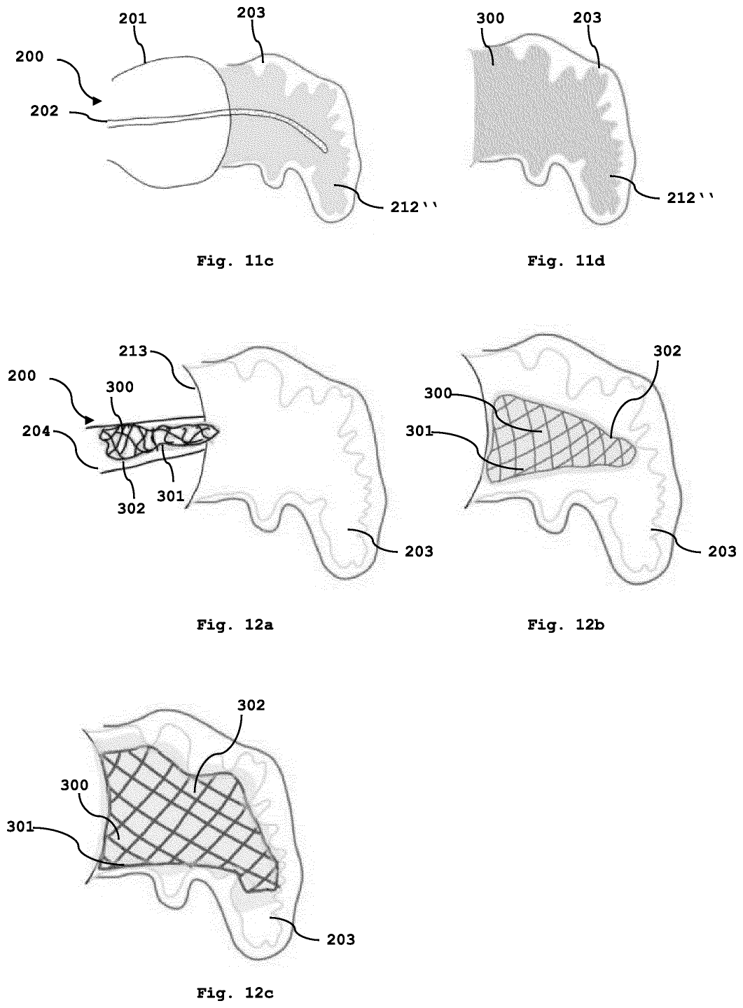

FIG. 12a shows a cavity 203 with a device 200 arranged at the ostium 213 of the cavity 203. Inside a lumen 204 of the device 200 is arranged an implant 300. The present implant comprises a mesh 301 that is coated with a hydrogel 302. The implant 300 is shown in a collapsed state wherein the implant may be placed inside the lumen 204 of a delivery device 200.

FIG. 12b shows the implant 300 after removal from the delivery device 200 and placed inside the cavity. The implant 200 is expandable via a shape memory effect of the mesh 301. Here, the implant 300 has expanded from its collapsed state but has not reached a fully expanded state yet (see FIG. 12c).

FIG. 12c shows the implant 300 in its fully expanded state. In addition, the hydrogel coating 302 is swollen and provides adhesion, either alone by hydrostatic pressure or by chemical bonding, to the inner wall of the cavity 203.

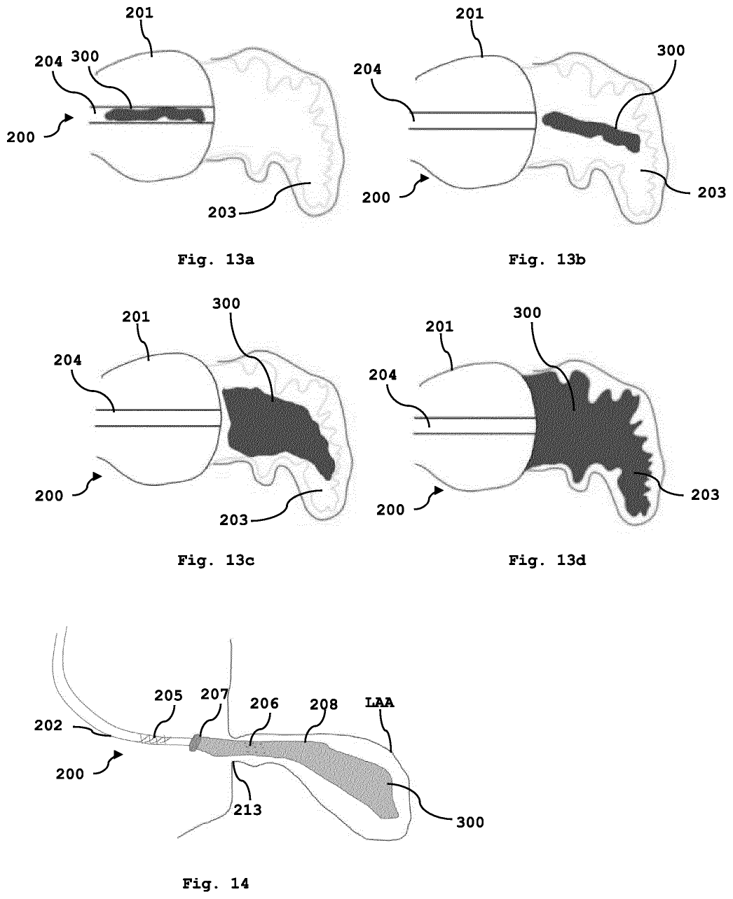

FIGS. 13a-13d show the implantation of an implant 300. The method of implantation is similar to the one illustrated in FIGS. 12a-12d.

FIG. 13a, however, shows the device comprising a sealing member 201 configured as a balloon. A lumen 204 is arranged within the balloon 201. The implant 300 is arranged within the lumen 204. The implant is configured as super-absorbent polymer that swells and forms a hydrogel when exposed to an aqueous liquid such as blood or saline.

FIG. 13b shows the implant 300 after having been pushed out of the lumen 204 and thus placed within the cavity 203. Here, the implant 300 is illustrated before having substantially swollen.

FIG. 13c shows the implant 300 after one minute in the cavity 203 and exposure to humidity. As a consequence, the implant 300 swells and its size increases. It would be possible to configure the implant 300 swell after a short or a longer time period as well.

As shown in FIG. 13d, the implant 300 in a final state wherein it occludes the cavity 203. Moreover, the super-absorbent hydrogel is mechanically flexible and as such as adapts to the shape of the cavity 203, wherein liquid, where present, may be absorbed by the implant 300. Thus, the cavity 203 is entirely filled by the implant 300.

FIG. 14 shows an alternative embodiment of an implant 300 with a delivery device 200. The implant 300 comprises a bag 208 whose volume significantly exceeds the volume of the cavity 203. In addition, the bag 208 is made of a material that is mechanically very flexible, for example polyethylene, polyurethane, ePTFE, silicone, and/or blends and co-polymers of these materials. The bag may be oversized with respect to the size of the cavity in which it is implanted and very compliant such as to adapt to the shape of the cavity. Such a bag is advantageous in that it may not need to be aligned with the cavity before implantation. The bag 208 further comprises pores 206 which are arranged at a proximal region of the implant 300 and configured to be placed in the region of the ostium 213 of the cavity. However, it would also be conceivable to configure the bag 300 without such pores 206 or to arranged the pores 206 at a different position of the bag 208. The delivery device 200 is connected to the bag 208 via a detachable connector 207 which comprises a screw mechanism. The delivery device 200 further comprises a mixing chamber 205 to mix a two-component adhesive that is transported through a transmission line 202. Thus, the mixed two-component adhesive can be transported through the transmission line into the bag 208. Additionally or alternatively, the bag 208 may be filled with a foam or a resin. The increased volume of the bag 208 fills the cavity 203, wherein the flexible material of the bag 208 allows the bag to conform to the shape of the cavity 203. Adhesive may perfuse through the pores 206 such as to provide adhesion to the region of the ostium 213. Subsequently, the adhesive may harden, either via an external curing mechanism or spontaneously, such as to form a hardened implant 300 attached to the cavity. The implant 300 may be detached from the delivery device 200 by detaching of the detachable connector 207. The detachment mechanism may be a screw mechanism, wherein an inner thread in the detachable connector 207 is in operative connection to an outer thread of the delivery device 200. Alternatively, an inner catheter of the delivery device 200, configured to form a self-sealing connection with the detachable connector 207, may be retracted from the bag 208.

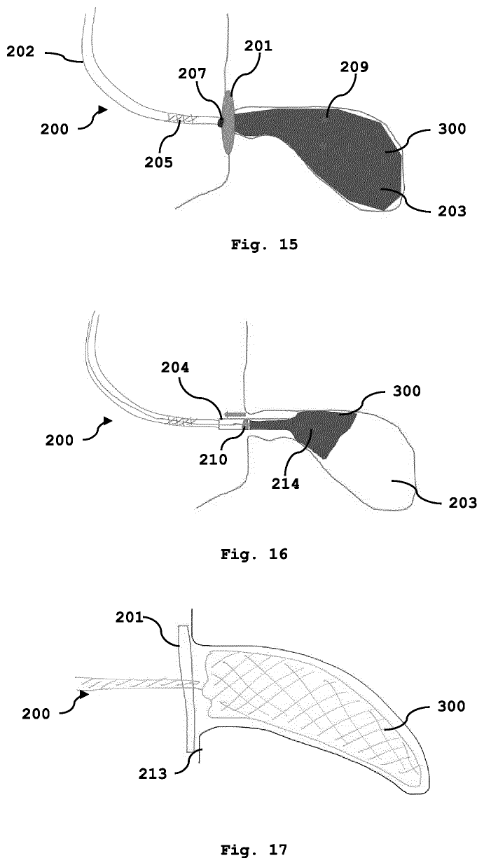

FIG. 15 shows an implant 300 being assembled in-vivo by means of a delivery device 200. The delivery device 200 is similar to the one shown in FIG. 14 and comprises transmission line 202 with a mixing chamber 205. A detachable connector 207 is attached to a distal end of the delivery device 200. A sealing member 201, here in the form of an expandable sealing disk, is reversibly attached to the delivery device 200 via the detachable connector 207. The sealing disk 201 provides a sealed closure to the cavity 203, such that an adhesive composition may be injected in the cavity 203 through the transmission line 202. The adhesive may be any adhesive composition known in the art, in particular any adhesive composition disclosed herein. Particularly preferably, the adhesive composition is a two-component resin or foam. Furthermore, the sealing disk is connected to two anchors 209 placed within the cavity 203. During adhesive delivery and hardening, the sealing disk 201 forms a barrier between the adhesive in the cavity 203 and the rest of the patient's body, for example the patient's left atrium. After hardening of the adhesive the anchors 209 form an irreversible connection between the adhesive plug in the cavity 203 and the sealing disk 201, together forming the implant 300.

FIG. 16 shows the implantation of an implant 300 using a delivery device 200. Here, the implant 300 is configured as a sponge with microchannels 214. The implant can be delivered in a capsule 204, in which it is constrained to a collapsed size, and pushed out the capsule 204 with a plunger 210, in particular through withdrawal of sheath. In addition, an adhesive composition may be deployed through the delivery device and the microchannels 214 of the implant 300 in order to attach the implant to an inner wall of the cavity 203. Any adhesive as disclosed herein is suitable to be combined with the implant and method shown here.

FIG. 17 shows schematically a general principle of occluding a cavity 203 according to the invention. A temporary occluding frame 201 may be employed to seal the ostium 213. An implant 300 formed of a curable material is inserted in the cavity 203 by means of a delivery device and becomes solid via a curing mechanism. Any curing mechanism disclosed herein is suitable to cure the curable material, in particular photocuring by exposure to electromagnetic radiation (UV light, IR light, visible light, X-rays). Additionally or alternatively, a chemical activator such as a cross-linking agent and/or photoinitiator may be employed.

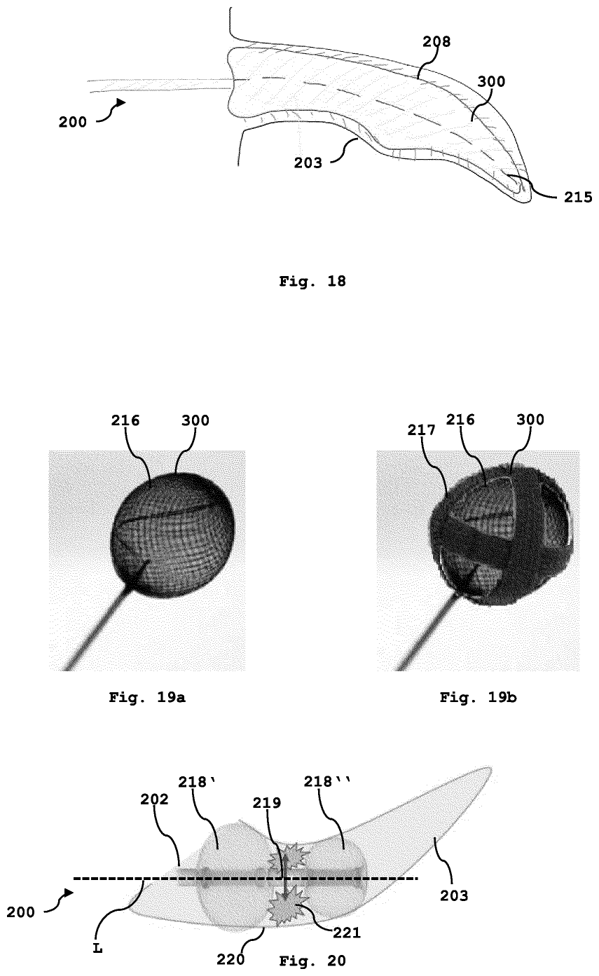

FIG. 18 shows an alternative general principle of occluding a cavity 203. A flexible balloon 208 may be inserted in the cavity 203 and filled with an adhesive composition such as to form an implant 300. In addition, a thread 215 may be used to control the length of the implant 300 before curing.

FIG. 19a shows an alternative embodiment of an implant 300. The implant is formed as a spherical braided mesh 216 and may or may not be filled with a viscous gel and/or an adhesive. Such a gel/adhesive may seal the braided mesh such that blood cannot penetrate it. Furthermore, the gel/adhesive may provide adhesion to tissue. The implant 300 is mechanically flexible and can adapt its shape to the shape of a cavity, in particular elliptical shapes. The braided mesh 216 is adapted in its mesh size to avoid leaks of a viscous gel inside the implant 300. Preferably, the size of the mesh is in the range of 10 μm to 500 μm, particularly preferably in the range of 50 to 100 μm, even more preferably in the range of 60 to 80 μm. In addition, the braided mesh 216 is adapted to enhance tissue ingrowth. Thus, the implant 300 shown here may be completely overgrown by tissue such as to occlude a cavity. The implant 300 shown here with a spherical shape is particularly advantageous because it can avoid issues with the alignment between the ostium and the implant 300 and simplify the approach angle during implantation. For example, the spherical shape may be introduced and remain in a cavity at any orientation angle. In addition, the spherical shape may conform to the shape of the cavity if the spherical shape were slightly oversized, i.e. larger than, relative to the cavity.

FIG. 19b shows an implant 300 similar to the implant of FIG. 19b. The implant 300 here additionally comprises a stripes 217 of fabric sutured to around the braided mesh 216. A light-activatable gel is coated on the stripes 217. The stripes 217 are sufficiently mechanically elastic such as to adjust to the deformation of the mesh 216.

FIG. 20 shows a distal end of a delivery deliver device 200. The delivery device 200 comprises a transmission line 202. A first balloon 218′ and a second balloon 218″ are attached to the transmission line 202 at a distance from each other along the longitudinal axis L of the transmission line 202. The transmission line 202 further comprises an opening 2019 located between the first and the second balloon 218′, 218″ through which an adhesive may be injected. Thus, the delivery device 200 may be inserted in a cavity 203, as shown here, and the balloons 218′, 218″ may be inflated to create a sealed enclosure 220 within the cavity 203. The sealed enclosure 220 can be filled with an adhesive such as to create an implant formed by an adhesive plug. Preferably, the adhesive is a two-component adhesive that cures upon mixing. Additionally or alternatively, the adhesive may be curable by light 221. After formation of the implant, the delivery device 200 and balloons 218″ the 218′, are retracted. Alternatively, one or both of the balloons 218′, 218″, preferably the distal balloon 218″, may be detachable from the delivery device 200 and remain in the body such as to form part of the implant.

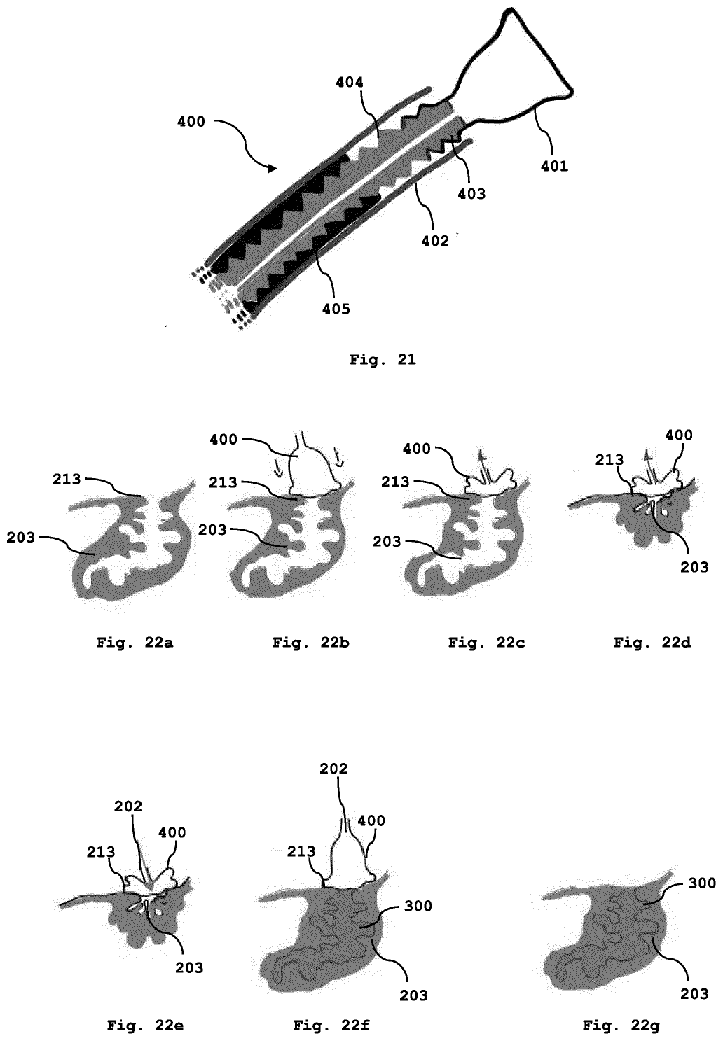

FIG. 21 shows a treatment device 400 according to the invention. The treatment device 400 may be configured as a delivery device as disclosed herein (200, see FIG. 13-18, for example). The treatment device 400 comprises a suction hood 401, preferably made of silicone. Alternatively, any biocompatible polymer is suitable. The suction hood 401 is connected to a silicone tube 403 which is configured with an external thread 404. The tube is mechanically adapted to not collapse if a vacuum is applied to its lumen while normal pressure is applied to its surrounding. The external thread 404 of the silicone tube 403 is in operative connection with an internal thread 405 fixedly connected to a sheath 402. By means of the internal thread 404 and the external thread 405, the suction hood may be deployed or retracted. Alternatively, a conventional sliding mechanism known in the art may be employed for movement of the suction hood 401 relative to the rest of the treatment device 400. By attaching to tissue via vacuum, the treatment device 400 may advantageously stabilize itself and/or a separate delivery device to a tissue to remove blood and/or insert another material or implant.

FIGS. 22a-22g schematically show a possible treatment with a treatment device 400 as shown in FIG. 21.

FIG. 22a shows a cavity 203 to be treated by occlusion, in the present case a left atrial appendage (LAA).

In FIG. 22b, a treatment device 400 is placed on the ostium 213 of the cavity.

Subsequently, as shown in FIG. 22c, a vacuum is applied by means of the treatment device.

As a consequence, as shown in FIG. 22d, the cavity 203 collapses due to the underpressure caused by the vacuum and the treatment device 400.

As mentioned with respect to the treatment device 400 of FIG. 21, the treatment device 400 may additionally be configured as a delivery device. Here, the treatment device 400 further comprises a transmission line 202 configured to deliver an adhesive composition (see FIG. 22f).

FIG. 22f shows the cavity 203 after it has been filled with an adhesive composition that subsequently forms an implant 300. The adhesive composition particularly preferably comprises a cross-linkable hydrogel. The pressure inside the cavity 203 here is the same as a physiological value without any intervention, i.e. substantially identical as the blood pressure for example shown in FIG. 22a. However, it would be possible to use a pressure higher or lower than physiological pressure. The adhesive is curable at body temperature and thus hardens spontaneously. It would be possible to use any adhesive disclosed herein, in particular adhesives that are curable by light irradiation, humidity, additives, or mixture of two or more components.

Subsequently, the implant 300 fills and occludes the cavity, as shown in FIG. 22g.

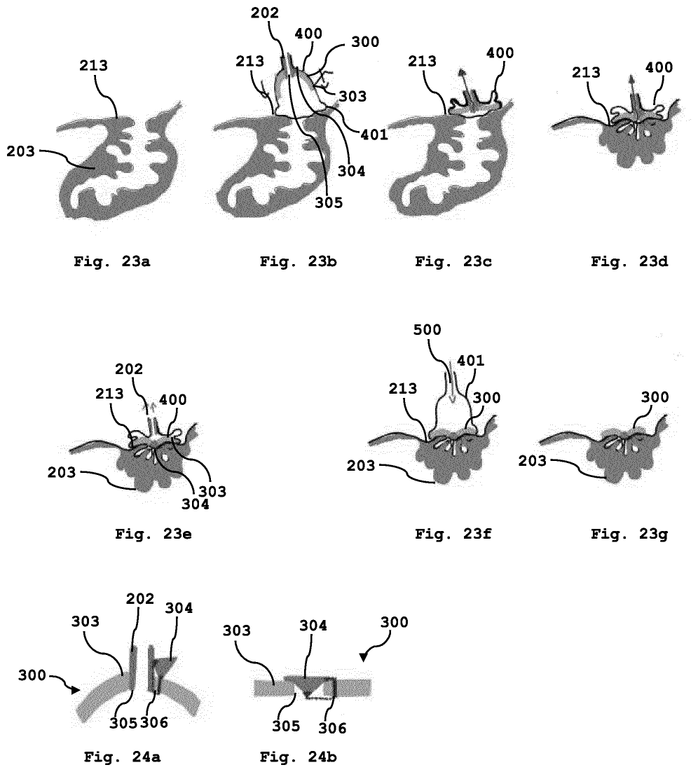

FIGS. 23a-23g show a method similar to the method shown in FIGS. 22a-23g.

FIG. 23a a cavity 203, here a left atrial appendage (LAA).

FIG. 23b shows a step substantially identical to the step shown in FIG. 22b. However, the treatment device 400 is additionally configured to carry an implant 300. The implant 300 comprises adhesive an patch made of expanded polytetrafluoroethylene (ePTFE) 303 and a silicone plug 304. Any adhesive disclosed herein is suitable to be included in the adhesive patch. The plug 304 is arranged in proximity to an opening 305 and is adapted to close the opening 305. The treatment device 400 further comprises a transmission line 202 that extends through the opening 305 in the patch 303 and is in fluid communication with an inner area of the suction hood 401.

The steps illustrated in FIGS. 23c and 23d are substantially identical to the steps of FIGS. 22c and 22d.

As shown in FIG. 23e, the transmission line 202 is subsequently retracted such that is no longer extends through the opening 305 of the patch 303. The silicone plug 304 automatically closes the opening 305 by a spring action (see FIGS. 24a and 24b). Accordingly, the cavity 203 is sealed under vacuum.

FIG. 23f shows that the inner volume of the suction hood 401 is filled with a saline solution 500 to fill the void and increase the pressure. In parallel, the implant forms and adhesive bond with ostium 213 of the cavity 203 such as to permanently seal the cavity.

FIG. 23g shows the cavity 203 being closed after removal of the treatment device 400.

FIG. 24a shows the implant 300 as illustrated in FIGS. 23b. The implant 300 comprises a patch 303 and a plug 304. The plug 304 is held by an elastic band 306 under tension as the transmission line 202 of the delivery device occludes the opening 305. Here, the plug 304 is arranged on a proximal side of the patch 303, but it would be conceivable to arranged the plug 304 on a distal side as well.

FIG. 24b shows the implant 300 of FIG. 24a after retraction of the transmission line 202. Due to the elastic force of the elastic band 306, the plug 304 is pulled into the opening 305 and occludes the same.

1-35. (canceled)

36. A medical implant that is adapted to close a defect or a cavity, wherein the medical implant comprises an occlusion device,
the medical implant comprises two states and is adapted to, in a first state, be deployed to a defect site,
where it can be brought into a second state by an activation mechanism and is adapted to close said defect or cavity in said second state.

37. The medical implant according to claim 36, wherein the medical implant comprises at least one peripheral disk.

38. The medical implant according to claim 37, wherein the at least one peripheral disk is adapted to mechanically anchor the occlusion device.

39. The medical implant according to one of the claims 37, wherein the occlusion device is connected or connectable to at least one peripheral disk by an interconnector.

40. The medical implant according to claim 36, comprising a braided structure, wherein the medical implant is adapted such that it has a substantially elongated shape in its first state, and comprises at least one disk that extends orthogonally from its longitudinal axis in its second state.

41. The medical implant according to claim 36, wherein the medical implant comprises an adhesive composition, wherein in the first state, the adhesive composition is contained in the medical implant and the medical implant is adapted to release said adhesive composition in its second state.

42. The medical implant according to claim 41, wherein the medical implant comprises at least two cavities and the adhesive composition comprises at least two components that are individually disposed in the at least two cavities.

43. The medical implant according to claim 41, wherein the adhesive composition is adapted to be gradually curable.

44. The medical implant according to claim 36, wherein the adhesive composition is adapted to be curable by exposure to electromagnetic radiation.

45. The medical implant according to claim 41, wherein the medical implant comprises an expandable material.

46. The medical implant according to claim 45, wherein the expandable material is adapted to expand upon deployment and occlude said defect upon expansion.

47. The medical implant according to claim 36, wherein the medical implant comprises a central diameter, and two peripheral diameters, the central diameter being smaller than the peripheral diameters, such that the medical implant comprises a dumbbell shape.

48. The medical implant according to claim 36, wherein the occlusion device comprises pericardial tissue.

49. The medical implant according to claim 36, wherein the occlusion device comprises a material that is flexible in the first state and becomes stiff in the second state.

50. The medical implant according to claim 36, wherein the medical implant comprises a distal end and a proximal end, the distal end being stiffer than the proximal end such that the distal end is adapted to serve as a cap towards the left atrium.

51. The medical implant according to claim 36, wherein the implant comprises a dead-end cavity inside the medical implant.

52. The medical implant according to claim 36, wherein the implant is self-expanding.

53. The medical implant according to claim 36, wherein the implant is at least partially transparent to visible, ultraviolet and/or infrared light along a longitudinal axis of the implant.

54. The medical implant according to claim 36, wherein the implant has one of a size and a shape that at least partially substantially corresponds to a human left atrial appendage.

55. The medical implant according to claim 36, wherein the implant has a first and a second section along a longitudinal axis, wherein the first section has a larger cross-section in a plane perpendicular to the longitudinal axis than the second section.

56. The medical implant according to claim 36, wherein the implant has a flat shape.

57. The medical implant according to claim 36, wherein the implant has at least one section with a diameter in the range of 10-25 mm.

58. A delivery device carrying a medical implant according to claim 36.

59. A method of closing a defect in a patient characterized in that the defect is closed with a medical implant according to claim 36.

60. A method of closing a cavity in a patient comprising a step of sealing an area outside of the cavity, and applying a negative pressure to the cavity such as to collapse the cavity, further comprising at least one of the steps of filling the collapsed cavity with an adhesive and sealing the collapsed cavity permanently.
